(19) United States
(12) Patent Application Publication
(10) Pub. No.: US 2024/0299279 A1
(43) Pub. Date: Sep. 12, 2024

(54) TRANSPARENT COSMETIC COMPOSITION WITH ASCORBIC ACID AND A SPECIFIC CATIONIC POLYMER

(71) Applicant: L'OREAL, Paris (FR)

(72) Inventors: Naima YOUSFI, Chevilly la Rue (FR); Muriel ISOIR-INGREZ, Chevilly la Rue (FR); Anne FALIP, Chevilly la Rue (FR); Laurence SEBILLOTTE-ARNAUD, Chevilly la Rue (FR)

(73) Assignee: L'OREAL, Paris (FR)

(21) Appl. No.: 18/572,229

(22) PCT Filed: Jun. 28, 2022
(86) PCT No.: PCT/EP2022/067768

§ 371 Date: Dec. 20, 2023

(30) Foreign Application Priority Data
Jun. 28, 2021 (FR) 2106896

(51) Int. Cl. A61K 8/67; A61K 8/73; A61Q 19/02; A61Q 19/08

(52) U.S. Cl. CPC A61K 8/676; A61K 8/731; A61K 8/737; A61Q 19/02; A61Q 19/08; A61K 2800/262; A61K 2800/52

(57) ABSTRACT

The present invention relates to a cosmetic composition comprising:
at least 30% by weight of water with respect to the total composition weight,
at least 5% by weight of ascorbic acid with respect to the total composition weight,
at least one cationic polymer chosen from cationic galactomannan gums and cationic cellulose polymers modified by groups including at least one linear fatty chain,
   said composition having a pH between 5 and 7, preferably between 5.5 and 6.5.

It also relates to a cosmetic product for the care of keratin materials using this composition.

The present invention relates to an aqueous transparent cosmetic composition, comprising ascorbic acid and at least one cationic polymer chosen from cationic galactomannan gums and cationic cellulose polymers modified by groups including at least one linear fatty chain.

In cosmetic applications, it is routine to add ascorbic acid (vitamin C) as a skin regenerating agent thanks to the stimulation of collagen synthesis responsible for skin firmness, or as a depigmenting agent, as this vitamin lowers the production of melanin responsible for brown spots. Ascorbic acid is also known for the antioxidant properties thereof.

However, when ascorbic acid is introduced into an aqueous medium (such as emulsions or serums) at high levels (i.e., for example greater than 1% by weight, preferably greater than 5% by weight), the formulation thereof is difficult to carry out, as it becomes unstable and changes color dramatically. This color change can range up to a brown coloration, which is unacceptable for the consumer.

For this reason, serum or solution type aqueous compositions, which comprise ascorbic acid in the form of powder to be solubilized extemporaneously, are found on the market. The drawback of this type of products is that vitamin C eventually degrades in the aqueous medium. These products thus lose their appeal as consumers prefer, for practical reasons, serums with no prior handling, with the guarantee of color stability and with no chemical degradation. Consumers also prefer a product with a low capacity (for example 10 ml), which enables quick use.

There is therefore a need for an aqueous cosmetic composition comprising ascorbic acid which is stable, i.e., which stays the same color as originally (generally white) and the same texture, and which does not degrade the active agent.

Surprisingly, the Applicant demonstrated that specific cationic polymers make it possible to limit degradation of the color of compositions comprising ascorbic acid, over time and temperature, while preserving the stability of the compositions obtained.

Thus, the present invention relates to a cosmetic composition comprising:
at least 30% by weight of water with respect to the total composition weight,
at least 5% by weight of ascorbic acid with respect to the total composition weight,
at least one cationic polymer chosen from cationic galactomannan gums and cationic cellulose polymers modified by groups including at least one linear fatty chain,
   said composition having a pH between 5 and 7.

The invention also relates to a cosmetic method for the care of keratin materials, preferably the skin, comprising the application of a composition according to the invention on said keratin materials.

Preferably, the composition according to the invention is an aqueous solution, more preferably an aqueous solution of slightly gelled appearance. Preferably, the composition according to the invention is single-phase. This composition can be referred to as “serum”. The term “serum” denotes a composition of fluid texture, of slightly gelled appearance, that is flowing, and preferably concentrated with ascorbic acid (i.e., preferably at least 5% by weight of ascorbic acid).

Preferably, the composition according to the invention is substantially free of surfactants. The term “substantially free of surfactants” denotes that the composition according to the invention has a surfactant content less than or equal to 2% by weight, with respect to the total weight of the composition, preferably less than or equal to 1% by weight. The surfactants can preferably be peptizing agents, the concentration of which is between 0.1 and 2% by weight; they enable the solubilization of a small quantity of oil(s) or lipophilic compounds or perfume(s) (i.e., perfume or oil or lipophilic compound concentration between 0.05% and 2%).

### Aqueous Phase

The composition according to the invention comprises water, which forms the aqueous phase.

The water used can be sterile demineralized water and/or floral water such as rose water, cornflower water, chamomile water or linden water, and/or a spring or natural mineral water.

The composition preferably comprises at least 30% by weight of water with respect to the total weight of the composition.

The composition preferably comprises from 30% to 93% by weight of water with respect to the total weight of the composition, more preferably from 50% to 90%, even more preferably from 55% to 70%.

The aqueous phase can also comprise at least one water-soluble organic solvent, at 25° C.

Preferably, the water-soluble organic solvent is chosen from alcohols, polyols and mixtures thereof.

Among the alcohols, mention can be made of C₁-C₁₀, more preferably C₁-C₅, alcohols, such as ethanol, isopropanol, propanol and butanol.

The polyol is,, preferably chosen from polyols having 2 to 20 carbon atoms, preferably 2 to 6 carbon atoms, such as glycerol, diglycerol, propyleneglycol, isoprene glycol, dipropyleneglycol, butylene glycol, hexylene glycol, 1,3-propanediol, pentylene glycol, polyethyleneglycols having 2 to 200 ethylene oxide units and mixtures thereof.

Preferably, the water-soluble organic solvent is chosen from polyols, preferably glycerol and/or hexylene glycol and/or pentylene glycol.

Preferably, the composition comprises from 1% to 25% by weight of water-soluble organic solvent, with respect to the total weight of the composition, more preferably from 2% to 30% by weight, even more preferably from 5 to 20% by weight.

### Ascorbic Acid

The ascorbic acid according to the invention corresponds preferably to L-ascorbic acid, or vitamin C. Its structure is as in formula (I):

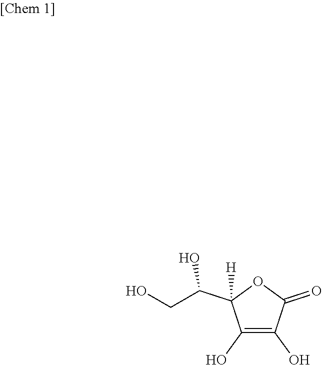 [SMILES: O=C1O[C@H]([C@@H](O)CO)C(O)=C1O]

The composition comprises at least 5% by weight of ascorbic acid with respect to the total weight of the composition, more preferably at least 7% by weight.

Preferably, the composition comprises from 5% to 30% by weight of ascorbic acid, with respect to the total weight of the composition, more preferably from 7% to 20% by weight.

As shown in the examples, surprisingly, the compositions according to the invention are perfectly transparent, comprise particularly 10% or 15% by weight of ascorbic acid, and have a good chemical stability and an acceptable color development after 2 months at 45° C. for 0.5% polyquaternium-67 and 2% cationic guar. This color development is acceptable as, scored on a color scale ranging from 0 (white, very light) to 9 (black, very dark), the compositions according to the invention have a score of 4, and this score is less than the reference composition comprising 10% by weight of ascorbic acid in water (scored 4.5, therefore greater than 4).

### Cationic Polymer Chosen from Cationic Galactomannan Gums and Cationic Cellulose Polymers Modified by Groups Including at Least One Linear Fatty Chain

The polymer according to the invention is chosen from cationic galactomannan gums (cationic guars) and cationic cellulose polymers modified by groups including at least one linear fatty chain (preferably polyquaternium-67).

Indeed, as shown in the examples, these polymers are more favorable for the cosmeticity and stability of the compositions.

The cationic polymer according to the invention can be a cationic galactomannan gum (cationic guar). The term “cationic galactomannan gum” denotes any galactomannan gum containing cationic groups and/or groups ionizable into cationic groups.

Galactomannans are polysaccharides essentially composed of galactose and mannose units, wherein the mannose units are bound by a 1-4-glycoside bond and galactose branching takes place by means of a 1-6 bridge to the mannose units. Each ring of the galactose or mannose units (or sugar units) carries three free hydroxyl groups available for the chemical reaction. Galactomannans are generally found in the endosperm of the grains of legumes such as guar or carob.

The preferred cationic groups are chosen from those including primary, secondary, tertiary and/or quaternary amine groups.

The cationic galactomannan gums used generally have a weight-average molecular weight between about 500 and 5×10⁶, and preferably between about 10³ and 3×10⁶.

The galactomannan groups suitable for use according to the present invention are for example gums including trialkyl (C₁-C₄) ammonium cationic groups. Preferably, 2% to 30% in number of the hydroxyl functions of these gums carry trialkylammonium cationic groups.

Among these trialkylammonium groups, mention can very particularly be made of trimethylammonium and triethylammonium groups.

Even more preferably, these groups represent from 5% to 20% by weight of the total weight of the modified galactomannan gum.

According to the invention, the cationic galactomannan gum is preferably a guar gum including hydroxypropyl trialkylammonium groups, more preferably a guar gum including hydroxypropyl trimethylammonium groups, i.e., a guar gum modified for example with 2,3-epoxypropyl trimethylammonium chloride.

These galactomannan gums in particular from guar modified by cationic groups are products already known per se and are for example described in the patents U.S. Pat. No. 3,589,578 and U.S. Pat. No. 4,031,307. Such products are moreover sold particularly under the trade names Jaguar EXCEL, Jaguar C13 S, Jaguar C 15, Jaguar C 17 and Jaguar C162 (Guar Hydroxypropyltrimonium Chloride) by Rhodia, under the name Amilan® Guar (Guar Hydroxypropyltrimonium Chloride) by Degussa, and under the name N-Hance® 3000 (Guar Hydroxypropyltrimonium Chloride) by Aqualon.

The cationic polymer according to the invention can alternatively be a cationic cellulose polymer modified by groups including at least one linear fatty chain. According to the present invention, a “cationic cellulose polymer” denotes any non-siliconized (comprising no silicon atoms) cellulose polymer containing cationic groups and/or groups ionizable into cationic groups, and preferably not containing anionic groups and/or groups ionizable into anionic groups.

The term “cellulose” polymer denotes according to the invention any polysaccharide compound having in the structure thereof at least 20 glucose residue chains joined by β-1,4 bonds. The cellulose polymer can be associative, i.e., having the structure thereof at least one C₈-C₃₀ fatty chain. The cationic cellulose polymers suitable for use preferably have a weight-average molecular weight (Mw) between about 5000 and 5·10⁶, preferably between about 10³ and 3·10⁶. Among the cationic celluloses, mention can more particularly be made of cellulose ethers including quaternary ammonium groups modified by groups including at least one linear fatty chain.

Among the quaternary celluloses, mention can be made in particular of quaternized celluloses modified by groups including at least one linear fatty chain, such as linear alkyl, linear or branched arylalkyl, linear alkylaryl, preferably linear alkyl, groups, these groups including at least 8 carbon atoms, particularly 8 to 30 carbon atoms, more preferably 10 to 24, or 10 to 14, carbon atoms; or mixtures thereof.

Preferably, mention can be made of quaternized hydroxyethylcelluloses modified by groups including at least one linear fatty chain, such as linear alkyl, linear arylalkyl, linear alkylaryl, preferably linear alkyl, groups, these groups including at least 8 carbon atoms, particularly 8 to 30 carbon atoms, more preferably 10 to 24, or 10 to 14, carbon atoms; or mixtures thereof.

Preferably, mention can be made of hydroxyethylcelluloses of formula (lb):

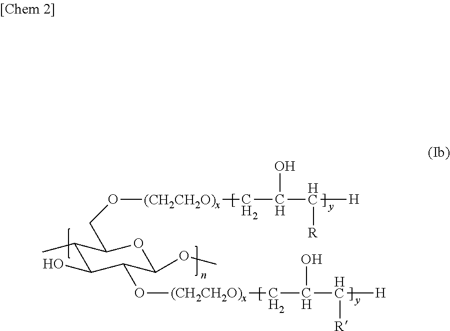

wherein:
R represents an ammonium group RaRbRcN⁺-, Q- wherein Ra, Rb, Rc, identical or different represent a hydrogen atom or linear, C1-C30, alkyl, preferably an alkyl and Q-represents an anionic counterion such as a halide such as chloride or bromide;
R′ represents an ammonium group R′aR'bR'cN⁺−, Q′− wherein R′a, R′b, R′c, identical or different represent a hydrogen atom or linear, C1-C30, alkyl, preferably an alkyl and Q′- represents an anionic counterion such as a halide such as chloride or bromide; preferably an alkyl;
   it being understood that at least one of the radicals Ra, Rb, Rc, R′a, R′b, R′c represents a linear, C8-C30, alkyl;
n, x and y, identical or different, represent an integer between 1 and 10000.

Preferably, in formula (lb), at least one of the radicals Ra, Rb, Rc, R′a, R′b, R′c represents a linear C8-C30 alkyl; preferably C10-C24, or C10-C14; mention can particularly be made of the dodecyl radical (C12). Preferably, the or the other radicals represent a linear C1-C4 alkyl, particularly methyl.

Preferably, in formula (lb), only one of the radicals Ra, Rb, Rc, R′a, R′b, R′c represents a linear C8-C30 alkyl; preferably C10-C24, or C10-C14; mention can particularly be made of the dodecyl radical (C12). Preferably, all the other radicals represent a linear C1-C4 alkyl, particularly methyl. More preferably, R can be a group chosen from —N⁺(CH₃)₃, Q′ and —N⁺(C₁₂H₂₅)(CH₃)₂, Q′⁻, preferably a group —N⁺(CH₃)₃, Q′⁻.

More preferably, R′ can be a group —N⁺((C₁₂H₂₅)(CH₃)₂, Q′⁻.

The nitrogen percentage can be vary from 0.1 to 10% by weight with respect to the total polymer weight, preferably from 0.2 to 5% by weight and more preferably from 0.5 to 3% by weight.

Mention can particularly be made of polymers having the INCI names:
Polyquaternium-24, such as the product QUATRISOFT LM 200®, marketed by AMERCHOL/DOW CHEMICAL;
PG-Hydroxyethylcellulose Cocodimonium Chloride, such as the product CRODACEL QM®;
PG-Hydroxyethylcellulose Lauryldimonium Chloride (C12 alkyl), such as the product CRODACEL QL® and
PG-Hydroxyethylcellulose Stearyldimonium Chloride (C18 alkyl) such as the product CRODACEL QS®, marketed by CRODA.

Mention can also be made of hydroxyethylcelluloses of formula (lb) wherein R represents trimethylammonium halide and R′ represents dimethyldodecylammonium halide, preferably R represents trimethylammonium chloride Cl—,(CH3)3N+− and R′ represents dimethyldodecylammonium chloride Cl—,(CH3)2(C12H25)N+−. This type of polymer is known under the INCI name Polyquaternium-67; as commercial products, mention can be made of SOFTCAT POLYMER SL® polymers such as SL-100, SL-60, SL-30, SL-5 and SX-1300X from AMERCHOL/DOW CHEMICAL.

More particularly, the cationic cellulose polymer is chosen from hydroxyethyl celluloses having reacted with a trimethyl ammonium epoxide and a lauryl dimethyl ammonium epoxide (INCI name POLYQUATERNIUM-67).

It is preferably marketed under the name Softcat Polymer SL-100 or Softcat Polymer SX-1300X by Amerchol.

Preferably, the cationic polymer is chosen from guar gums comprising trialkylammonium cationic groups and quaternized hydroxyethylcelluloses modified by groups including at least one linear fatty chain; preferably, from guar gums modified by a salt of 2,3-epoxypropyl trimethylammonium and the hydroxyethylcelluloses of formula (lb) wherein R represents trimethylammonium halide and R′ represents dimethyldodecylammonium halide

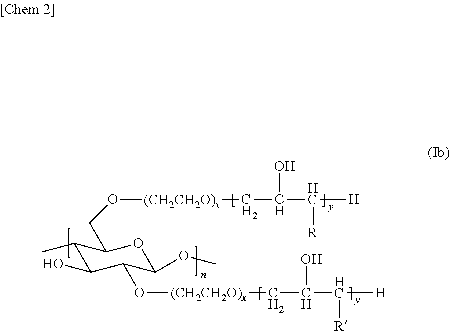

and preferably from Guar Hydroxypropyl Tri-Methyl Ammonium chloride and hydroxyethyl celluloses having reacted with a trimethyl ammonium epoxide and a lauryl dimethyl ammonium epoxide (INCI name polyquaternium-67).

The total active substance content of the cationic polymer(s), present in the composition according to the invention, ranges preferably from 0.05 to 5% by weight, preferably from 0.3 to 3% by weight, and more preferably from 0.4 to 2% by weight with respect to the total weight of the composition.

### pH of the Composition

The pH of the composition according to the invention is 5.0 to 7.0. Advantageously, the pH of the composition is between 5.5 and 6.5.

Indeed, such a pH limits the degradation of the color of the ascorbic acid, and thus limits the transformation of a colorless composition into a composition which turns yellow to brown over time. When the pH is less than 5, the composition turns brown, and, scored on a color scale ranging from 0 (white, very light) to 9 (black, very dark), it has a score of above 8.

Preferably, the cosmetic composition according to the invention comprises at least one base.

The base is particularly used to increase the final pH of the composition to between 5.0 and 7.0, preferably between 5.5 and 6.5.

The base can be chosen from mineral bases such as for example alkaline metal hydroxides, sodium hydroxide or potassium hydroxide.

Preferably, the base of the composition is an alkaline metal hydroxide, preferably sodium hydroxide or potassium hydroxide, preferably sodium hydroxide. Indeed, these bases are more advantageous than nitrogenous bases, such as triethanolamine, on color stabilization (i.e., less yellowing).

Preferably, the base of the composition according to the invention is present in a base:ascorbic acid weight ratio ranging from 0.22 to 0.30.

The composition can also comprise one or more additive(s) conventionally used in cosmetic compositions such as for example active agents, sequestering agents, glycols, preservatives or perfumes.

Preferably, among the usable active agents, adenosine, salicylic acid, glycolic acid, niacinamide (Vitamin B3), tocopherol or perfumes are preferred.

The composition according to the invention can comprise at least one sequestering agent. Among the preferred sequestering agents, mention can be made of ethylene diamine disuccinic acid.

Ethylene diamine disuccinic acid is a compound of formula (II):

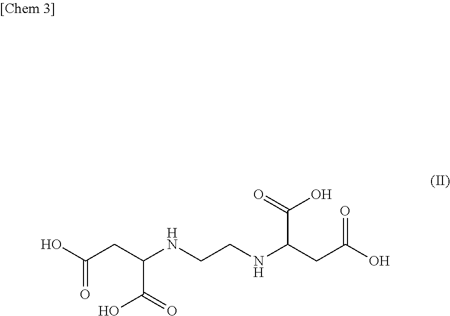 [SMILES: O=C(O)CC(NCCNC(CC(=O)O)C(=O)O)C(=O)O]

Preferably, the ethylene diamine disuccinic salt is chosen from alkaline metal salts, such as potassium salts and sodium salts, ammonium salts, and amine salts. The alkaline metal salts of ethylene diamine acid are more particularly preferred.

Preferably, the ethylene diamine disuccinic acid salt used according to the invention is trisodium ethylene diamine disuccinate.

Such a compound is for example that marketed under the name Natriquest® E30 by Innospec Active Chemicals, or that marketed under the name Octaquest E30® by Octel Performance Chemicals.

Preferably, the composition according to the invention comprises from 0.01% to 2.5% by weight, preferably from 0.05% to 1.5% by weight, more preferably from 0.07% to 0.3% by weight of ethylene diamine disuccinic acid salt, with respect to the total weight of the composition.

It is understood that the ethylene diamine disuccinic acid salt corresponds to the active substance, also referred to as dry matter, content of ethylene diamine disuccinic acid salt introduced into the composition.

According to a specific embodiment, the ethylene diamine disuccinic acid salt can be introduced into the composition dissolved in water, particularly at a content ranging from 25% to 50% by weight, preferably from 35% to 40% by weight in water.

Such a compound is for example that marketed under the name Natriquest® E30 by Innospec Active Chemicals, at 37% by weight in water.

The composition according to the invention can comprise at least one glycol, preferably caprylyl glycol.

Preferably, the composition according to the invention comprises from 0.1% to 2% by weight, preferably from 0.2% to 1.5% by weight of glycol(s), with respect to the total weight of the composition.

The composition according to the invention can comprise at least one preservative. Among the preferred preservatives, mention can be made of hydroxyacetophenone, phenoxyethanol and mixtures thereof.

Preferably, the composition according to the invention comprises from 0.1% to 2% by weight, preferably from 0.5% to 1.5% by weight, more preferably from 0.7% to 1% by weight of preservative(s) with respect to the total weight of the composition.

Preferably, the composition according to this invention is transparent.

The term transparent composition according to the present invention denotes a composition with a turbidity value of less than 200 NTU, preferably less than 150 NTU, preferably less than 100 NTU. Preferably, the turbidity of compositions is equal to at least 1 NTU.

NTUs (nephelometric turbidity units) are units for measurement of the turbidity of a composition. The turbidity measurement is made, for example, with a model 2100P turbidity meter made by the Hach Company, the tubes used for the measurement being references AR397A cat 24347-06. The measurements are made at ambient temperature (from 20° C. to 25° C.).

Preferably, the composition is transparent and has a turbidity value equal to between 1 and 200 NTU, preferably between 1 and 150 NTU, preferably less than 100 NTU.

Finally, the invention also relates to a cosmetic method for the care of keratin materials, preferably the skin, comprising the application of a composition according to the invention. Concrete, yet non-limiting, examples, illustrating the invention, will now be provided.

### EXAMPLES

Unless stated otherwise, the pressure in the examples is atmospheric pressure. Unless specified otherwise, the percentages are expressed by weight in relation to the total weight of the composition (% w/w).

### Example 1: Influence of Cationic Polymers on Color Stabilization of an Aqueous Solution Comprising 10% by Weight of Vitamin C

### Comparative Examples

1/with polyquaternium-6 (MERQUAT 100 POLYMER from Nalco; poly dimethyl diallyl ammonium chloride in water at 40%, non-stabilized): Aqueous solutions are prepared comprising 10% by weight of vitamin C, from 1% to 40% by weight of polyquaternium-6, and having a pH equal to 6, the pH having been adjusted with sodium hydroxide (NaOH).

A stability test is performed on the solutions for 15 days, 1 month and 2 months at 45° C.

The results show that the increase in the polyquaternium-6 concentration goes against the expected effect, i.e., there is degradation of the color which is increasingly dark. The higher the polyquaternium-6 concentration, the more visible the color degradation (dark) is.

2/with Cassia Hydroxypropyltrimonium Chloride (ClearHance C from Ashland):
Aqueous solutions are prepared comprising 10% by weight of vitamin C, from 0.5% to 4% by weight of Cassia Hydroxypropyltrimonium Chloride, and having a pH equal to 6, the pH having been adjusted with sodium hydroxide (NaOH).

A stability test is performed on the solutions for at least 1 month, either at ambient temperature or at 45° C.

The results show that the increase in the Cassia Hydroxypropyltrimonium Chloride concentration goes against the expected effect, i.e., there is degradation of the color which is increasingly dark.

3/with N-acetyl-D-glucosamine polysaccharide (KIONUTRIME CSG from Kitozyme):
Aqueous solutions are prepared comprising 10% by weight of vitamin C, from 0.25% to 4% by weight of N-acetyl-D-glucosamine polysaccharide, and having a pH equal to 6, the pH having been adjusted with sodium hydroxide (NaOH).

A stability test is performed on the solutions for 1 month at 45° C.

The results show that the increase in the N-acetyl-D-glucosamine polysaccharide concentration goes against the expected effect, i.e., there is degradation of the color which is increasingly dark.

4/with cationic starch (starch hydroxypropyltrimonium chloride, PENCARE DP 1015® marketed by INGREDION):
Aqueous solutions are prepared comprising 10% by weight of vitamin C, from 0.5% to 4% by weight (0.5%; 1%; 2%; 4%) of cationic starch, and having a pH equal to 6, the pH having been adjusted with sodium hydroxide (NaOH).

A stability test is performed on the solutions for 2 month at 45° C.

The results show that there is a dose effect of the cationic starch concentration on color stability with respect to the reference without polymer. However, the formula obtained is turbid and does not solve the problem of the invention which is that of obtaining a transparent serum.

### Examples According to the Invention

1/with cationic galactomannan gum (Guar Hydroxypropyl Tri-Methyl Ammonium Chloride, hydroxypropyl guar hydroxypropyl trimonium chloride, Jaguar C 162 SGI from Rhodia):

Aqueous solutions are prepared comprising 10% by weight of vitamin C, from 0.5% to 4% by weight (0.5%; 1%; 2%; 4%) of cationic guar, and having a pH equal to 6, the pH having been adjusted with sodium hydroxide (NaOH).

A stability test is performed on the solutions for 2 months at 4° C., or 2 months at ambient temperature, or 2 months at 45° C.

The solutions obtained are all stable.

The solution comprising 2% of cationic guar, after storing for 2 months at 45° C., shows an acceptable vitamin C loss.

The results show that there is a dose effect of the cationic guar concentration on color stability with respect to the reference without cationic guar. The preferred concentration is between 0.5% and 4%.

2/with polyquaternium-67 (Softcat Polymer SL-100 from Amerchol):
Aqueous solutions are prepared comprising 10% by weight of vitamin C, from 0.5% to 4% by weight (0.5%; 1%; 2%; 4%) of polyquaternium-67, and having a pH equal to 6, the pH having been adjusted with sodium hydroxide (NaOH).

A stability test is performed on the solutions for 2 months at 4° C., or 2 months at ambient temperature, or 2 months at 45° C.

The solutions obtained are all stable, show an acceptable vitamin C loss.

The results show that there is a dose effect of the polyquaternium-67 concentration on color stability with respect to the reference without polymer. The preferred concentration is between 0.5% and 4%.

### CONCLUSION

The condition of a pH of 6 is not sufficient to stabilize the color of an aqueous vitamin C solution.

Not all the cationic polymers are effective in this functionality.

Associating sodium hydroxide or potassium hydroxide with a cationic polymer such as cationic galactomannan gums or specific cationic celluloses (polyquaternium-67) makes it possible to stabilize the color of the vitamin C solutions.

Only cationic galactomannan gums and specific cationic celluloses make it possible to combine color stabilization with obtaining a perfectly transparent serum.

### Example 2: Compositions According to the Invention

Formulas A to D according to the invention are prepared below according to the following protocol:
1—Disperse the cationic polymer (hydroxypropyl guar hydroxypropyl trimmonium chloride or polyquaternium-67) in a little water until a translucent and homogeneous (grain-free) gelled water appearance is obtained
2—Add ascorbic acid and water; the mixture then becomes perfectly transparent and colorless;
3—Adjust the pH to 6 with NaOH. A perfectly transparent gelled solution is obtained.

TABLE 1

| Ingredients | | | | |
|---|---|---|---|---|
| (% w/w) | Formula A | Formula B | Formula C | Formula D |
| Sodium hydroxide | Qs pH 6 | Qs pH 6 | Qs pH 6 | Qs pH 6 |
| (NaOH) | | | | |
| Ascorbic acid | 10 | 10 | 10 | 10 |
| Guar Hydroxypropyl | 1 | 2 | — | — |
| Tri-Methyl Ammonium | | | | |
| Chloride (Jaguar C | | | | |
| 162 SGI from Rhodia) | | | | |
| Polyquaternium-67 | — | — | 0.5 | 1 |
| (Softcat Polymer SL- | | | | |
| 100 from Amerchol) | | | | |
| Water | Qs 100 | Qs 100 | Qs 100 | Qs 100 |

### Example 3: Compositions According to the Invention

Formulas E and F according to the invention are prepared below according to the following protocol:

In a first beaker (phase A):
1. Introduce water (T>60° C.) and add the ingredient of phase Ain the following order: glycerin, caprylyl glycol, preservative(s), trisodium ethylenediamine dissuccinate, hexylene glycol and adenosine at 60° C.;
2. Once everything has dissolved and there are no more crystals, allow to cool.

In a second beaker (phase B):
1. Introduce water at ambient temperature and add ascorbic acid;
2. Adjust the pH to 5.8 with sodium hydroxide;
3. Add Polyquaternium-67 with moderate, lengthy stirring to ensure effective deployment of the polymer.

Mix the two beakers and make up to the QS with water if required.

TABLE 2

| Ingredients | | | |
|---|---|---|---|
| (% w/w) | Formula E | Formula F | Phase |
| Hexylene glycol | 5 | 5 | A |
| Sodium hydroxide (soda) | 3.33 | 3.33 | B |
| Ascorbic acid | 15 | 15 | B |
| Glycerin | 7 | 7 | A |
| Caprylyl glycol | Qs | Qs | A |
| Preservative(s) | Qs | Qs | A |
| Adenosine | — | 0.04 | A |
| Polyquaternium-67 (Softcat Polymer SL- | 0.5 | 0.5 | B |
| 100 from Amerchol) | | | |
| Trisodium ethylene diamine disuccinate | 0.2 | 0.2 | A |
| (Natriquest ® E30 from Innospec Active | (*0.074 in | (*0.074 in | |
| Chemicals, at 37% by weight of a.s. In | a.s.) | a.s.) | |
| water) | | | |
| Water | Qs 100 | Qs 100 | A |

a.s.: active substance

The compositions according to the invention have the following properties:
Maintenance of transparency at 1 (scale from 1 to 3:1 being transparent, 2 translucent, 3, opaque) over time and temperature (after 2 months at 4° C., ambient temperature and 45° C.);
No or acceptable slight gassing, i.e., release of CO2 due to the degradation of ascorbic acid over time and temperature (after 2 months at 4° C., ambient temperature and 45° C.);
No crystals observed macroscopically and microscopically over time and temperature (after 2 months at 4° C., ambient temperature and 45° C.); and
Physicochemical stability over time and at temperature (after 2 months at 4° C., ambient temperature and 45° C.).

1. A cosmetic composition comprising:
at least 30% by weight of water with respect to the total composition weight,
at least 5% by weight of ascorbic acid with respect to the total composition weight,
at least one cationic polymer chosen from cationic galactomannan gums and cationic cellulose polymers modified by groups including at least one linear fatty chain, said composition having a pH between 5 and 7.

2. The composition according to claim 1, which comprises from 30% to 93% by weight of water with respect to the total weight of the composition.

3. The composition according to claim 1, which comprises at least one water-soluble organic solvent, at 25° C., chosen selected from the group consisting of alcohols, polyols and mixtures thereof.

4. The composition according to claim 1, which comprises at least 7% by weight of ascorbic acid with respect to the total weight of the composition.

5. The composition according to claim 1, characterized in that wherein the cationic polymer is chosen from selected from the group consisting of guar gums comprising trialkylammonium cationic groups and quaternized hydroxyethylcelluloses modified by groups including at least one linear fatty chain.

6. The composition according to claim 1, which comprises from 0.05% to 5% by weight of cationic polymer active substance, with respect to the total weight of the composition.

7. The composition according to claim 1, characterized in that wherein the pH of the composition is between 5.5 and 6.5.

8. The composition according to claim 1, which comprises at least one base.

9. The composition according to claim 1, which comprises at least one additive chosen from active agents, sequestering agents, glycols, preservatives and perfumes.

10. The composition according to claim 1, which is transparent.

11. The cosmetic method for the care of keratin materials, comprising the application of a composition according to claim 1 onto said keratin materials.

12. The composition according to claim 3, wherein water-soluble organic solvent, at 25° C. is selected from the group consisting of C1-C10 alcohols and polyols having 2 to 20 carbon atoms, and mixtures thereof.

13. The composition according to claim 1, which comprises from 5% to 30% by weight of ascorbic acid, with respect to the total weight of the composition.

14. The composition according to claim 1, wherein the cationic polymer is selected from the group consisting of guar gums modified by a salt of 2,3-epoxypropyl trimethylammonium and hydroxyethylcelluloses of formula (Ib) wherein R represents trimethylammonium halide and R′ represents dimethyldodecylammonium halide

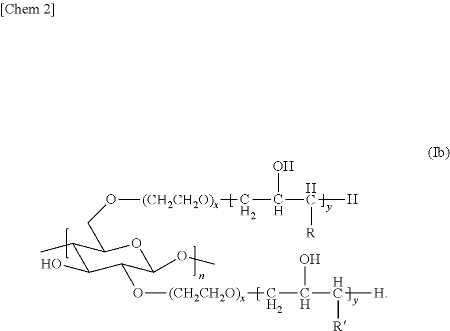

15. The composition according to claim 8, wherein the base of the composition is an alkaline metal hydroxide.

16. The composition according to claim 1, which comprises at least one sequestering agent chosen from ethylene diamine disuccinic acid and/or at least one glycol and/or at least one preservative.

17. The composition according to claim 2 which comprises at least one water-soluble organic solvent, at 25° C., chosen selected from the group consisting of from alcohols, polyols and mixtures thereof.

18. The composition according to claim 2, which comprises at least 7% by weight of ascorbic acid with respect to the total weight of the composition.

19. The composition according to claim 3, which comprises at least 7% by weight of ascorbic acid with respect to the total weight of the composition.

20. The composition according to claim 2, wherein the cationic polymer is selected from the group consisting of guar gums comprising trialkylammonium cationic groups and quaternized hydroxyethylcelluloses modified by groups including at least one linear fatty chain.
